(19) United States
(12) Plant Patent Application Publication
(10) Pub. No.: US 2023/0093797 P1
(43) Pub. Date: Mar. 23, 2023

(54) CANNABIS PLANT NAMED ZUCHI

(50) ;

(71) Applicant: Umami Worldwide, LLC, Las Vegas, NV (US)

(72) Inventor: Chase Martin, Las Vegas, NV (US)

(21) Appl. No.: 17/950,084

(22) Filed: Sep. 21, 2022

Related U.S. Application Data
(60) Provisional application No. 63/247,285, filed on Sep. 22, 2021.

(51) Int. Cl. A01H 6/28

(52) U.S. Cl. PLT/258

(57) ABSTRACT

The cannabis cultivar Zuchi can be briefly characterized by having a zigzag stem structure, medium length petioles, short internodal stacking, triangle-shaped flowers, and bulky flowers with little to no stem. It produces large towering colas up to 2′ in uninterrupted length. It has a short flowering time of less than 70 days, usually 60-65 days. Female plants having Zuchi as the male parent generally have deep purple and blue colorations in 50-75% of the progeny that are high yielding, greater than 50 grams/ft² under optimal conditions.

Latin name of the genus and species: Cannabis sativa.

Variety denomination: ‘ZUCHI’

### BACKGROUND OF THE INVENTION

This invention relates to a new and distinctive male cannabis cultivar designated as ‘ZUCHI’.

### TAXONOMY AND NOMENCLATURE

Cannabis, more commonly known as marijuana, is a genus of flowering plants that includes at least three species, Cannabis sativa, Cannabis indica, and Cannabis ruderalis as determined by plant phenotypes and secondary metabolite profiles. In practice however, cannabis nomenclature is often used incorrectly or interchangeably. Cannabis literature can be found referring to all cannabis varieties as “sativas” or all cannabinoid producing plants as “indicas”. Indeed, the promiscuous crosses of indoor cannabis breeding programs have made it difficult to distinguish varieties, with most cannabis being sold in the United States having features of both sativa and indica species.

Human cultivation history of Cannabis dates back 8000 years (Schultes, R E., 1970, Random thoughts and queries on the botany of Cannabis. Pages 11-38 in: C R B Joyce, and S H Curry eds., THE BOTANY AND CHEMISTRY OF CANNABIS. J. & A. Churchill. London, England). Hemp cloth recovered in Europe dates back 6000 years (Small, E, Beckstead, H D, and Chan, A, 1975, The evolution of cannabinoid phenotypes in Cannabis, ECONOMIC BOTANY 29(3):219-232). The written record of the pharmacologic properties of Cannabis goes back more than 4000 years (Ti, H. 2737 BC. NEI JING SU WEN HUANG TI, Yellow Emperor's Classic on Internal Medicine; referred to without citation in Small et al. 1975 Supra).

The taxonomy and nomenclature of the highly variable genus Cannabis (Emboden, W A, 1974, ECONOMIC BOTANY 28(3):304-310; Small, E and Cronquist, A, 1976, TAXON 25(4):405-435; Small E and Cronquist, A, 1977, TAXON 26(1):110; Hillig, K W and Mahlberg, P G, 2004, American Journal of Botany 91(6):966-975), remains in question. This is in spite of the fact that its formal scientific name, ‘Cannabis sativa L.’, assigned by Carolus Linneaus (Linnaeus, C, 1753, SPECIES PLANTARUM, 2:1027, Salvius, Stockholm, Facsimile edition, 1957-1959, Ray Society, London, U.K.), is one of the oldest established names in botanical history and is still accepted to this day. Another species in the genus, ‘Cannabis indica Lam.’ was formally named somewhat later (de Lamarck, J B, 1785, ENCYCLOPEDIE METHODIQUE DE BOTANIQUE, 1(2):694-695), but is still very old in botanical history. In 1785, Jean-Baptiste Lamarck published a description of a second species of Cannabis, which he named Cannabis indica. Lamarck based his description of the newly named species on plant specimens collected in India. C. indica was described as relatively short, conical, and densely branched, whereas C. sativa was described as tall and laxly branched (Schultes R. E. et al, 1974, Harvard University Botanical Museum Leaflets, 23:337-367). C. indica plants were also described as having short, broad leaflets whereas those of C. sativa were characterized as relatively long and narrow (Anderson L. C., 1980, Harvard University Botanical Museum Leaflets, 28:61-69). C. indica plants conforming to Schultes' and Anderson's descriptions may have originated from the Hindu Kush mountain range. Because of the often harsh and variable (extremely cold winters, and warm summers) climate of those parts, C. indica is well-suited for cultivation in temperate climates.

Three other species names were proposed in the 1800s to distinguish plants with presumably different characteristics (C. macrosperma Stokes, C. chinensis Delile, C. gigantean Vilmorin), none of which are accepted today, although the epithet “indica” lives on as a subspecies of C. sativa (‘C. sativa ssp. indica Lam.’, Small and Cronquist 1976 Supra).

In the 20th century, two new names were added to the liturgy of proposed ‘Cannabis species: C. ruderalis’ Janischevsky and a hybrid, x ‘C. intersita’ Sojak. (Small, E, Jui, P Y, and Lefkovitch, L P, 1976, SYSTEMATIC BOTANY 1(1):67-84; Small and Cronquist 1976 Supra). Further, numerous names have been proposed for horticultural variants of ‘Cannabis’ but as of 1976, “very few of these have been validly published as formal taxa under the International Code of Botanical Nomenclature” (Small and Cronquist 1976 Supra). Moreover, other recent work continues to focus on higher-order evolutionary relationships of the genus. Cannabis has been variously ascribed as belonging to mulberry family (Moraceae) (Engler, H G A, Ulmaceae, Moraceae and Urticaceae, pages 59-118 in: A. Engler and K. Prantl eds., 1889, DIE NATURLICHEN PFLANZENFAMILIEN 3(1). W. Engelmann, Leipzig, Germany; Judd, W S, Sanders, R W, and Donoghue, M J, 1994, HARVARD PAPERS IN BOTANY 5:1-51; Humphries, C J and Blackmore, S, A review of the classification of the Moraceae, pages 267-277 In: Crane and Blackmore 1989 id.); nettle family (Urticaceae) (Berg, C C, Systematics and phylogeny of the Urticales, pages 193-220, in: P. R. Crane and S. Blackmore eds., 1989, EVOLUTION, SYSTEMATIC, AND FOSSIL HISTORY OF THE HAMAMELIDAE,VOL. 2, HIGHER HAMAMELIDAE, Clarendon Press, Oxford, U.K.); and most recently in its own family with hops (Humulus), Cannabaceae, or hemp family (Sytsma, K J, et al, 2002, AMERICAN JOURNAL OF BOTANY 89(9): 1531-1546). While the work of Small and Cronquist 1976 Supra, seemed to effectively confine the genus to a single species with 2 subspecies (C. sativa s., C. s. indica), each with two varieties (C. s. s. var. sativa, C. s. s. var. spontanea; C.s. i. var. indica, C. s. i. var. Kafiristanica) largely on the basis of chemotaxonomy and interfertility of all forms, more recent work (Sytsma et al. 2002 Supra), proposes a two-species concept, resurrecting the binomial C. indica Lam. Since Sytsma et al. (2002) provides no key for discriminating between the species, the dichotomous key of Small and Cronquist (1976), which accounts for all forms in nature, whether wild or domesticated, is preferred to classify the characteristics of the plant.

The present invention relates to a new and distinct Cannabis cultivar designated “ZUCHI”.

ZUCHI is characterized as being a male cultivar having the following characteristics:

zig zag stem growth structure (FIGS. 2 and 4)

little to no purple coloration on the stem unless stressed. If stressed due to improper environment, feed, lighting etc, purple streaking in the stem and at the base of the petiole (FIG. 5) can be observed

Lemon pine cleaner scent on the stem rub

short internodal spacing—usually 1 inch or less between nodes

wavy leaf structure with rounded edge leaf serrations (FIGS. 1, 3, 6, 7, 9)

Usually a 5 finger leaf set up to 7 at maximum health, 4.5-5″ average length

Petiole length 2-2.5″

A darker green leaf (FIGS. 1, 5, 7) requiring high nitrogen feed when vegetative. We use calcium nitrate as 25% of the vegetative feed formula.

Vigorous growth—up to several inches per week

Significant lateral branching. The structure is in between a bush and a pine tree shape (FIG. 2).

Fast rooting. When cloned, roots appear in 7-10 days on average.

The plant stretches at 1.5-2 x its vegetative size during flowering

The pollen falls at 20-22 days after flowering

45 day flowering total

No purple coloration on the male plant at any time

No signs of pre flowering in during vegetative growth regardless of light schedule

No signs of intersex traits observed

Can be grown under a wide range of conditions, for example using an 18 h light/6 h dark schedule up to constant light

Prefers nutrients at 2.7-3.0 EC and a light point measurement of light intensity (PPFD) at 300-500 during vegetative growth and 700-1000 in bloom

### BRIEF SUMMARY OF THE INVENTION

The cannabis cultivar Zuchi can be briefly characterized by having a zigzag stem structure, medium length petioles, short internodal stacking, triangle-shaped flowers, and bulky flowers with little to no stem. It produces large towering colas up to 2′ in uninterrupted length. It has a short flowering time of less than 70 days, usually 60-65 days. Female plants having Zuchi as the male parent generally have deep purple and blue colorations in 50-75% of the progeny that are high yielding, greater than 50 grams /ft² under optimal conditions.

### BRIEF DESCRIPTION OF THE DRAWING

FIG. 1 is a view of the front of a leaf on a ZUCHI plant.

FIG. 2 is a view of a growing ZUCHI plant.

FIG. 3 is a view of the back of a leaf from a ZUCHI plant.

FIG. 4 is a view of the stem of a ZUCHI plant.

FIG. 5 is a view of a petiole of a ZUCHI plant.

FIG. 6 is a view of the back of a leaf from a ZUCHI plant.

FIG. 7 is a view of the front of a leaf on a ZUCHI plant.

FIG. 8 is a view of the stem of a ZUCHI plant.

FIG. 9 is a view of the back of a leaf from a ZUCHI plant.

FIG. 10 is a view of a flowering ZUCHI plant.

FIG. 11 is a view of a flowering ZUCHI plant.

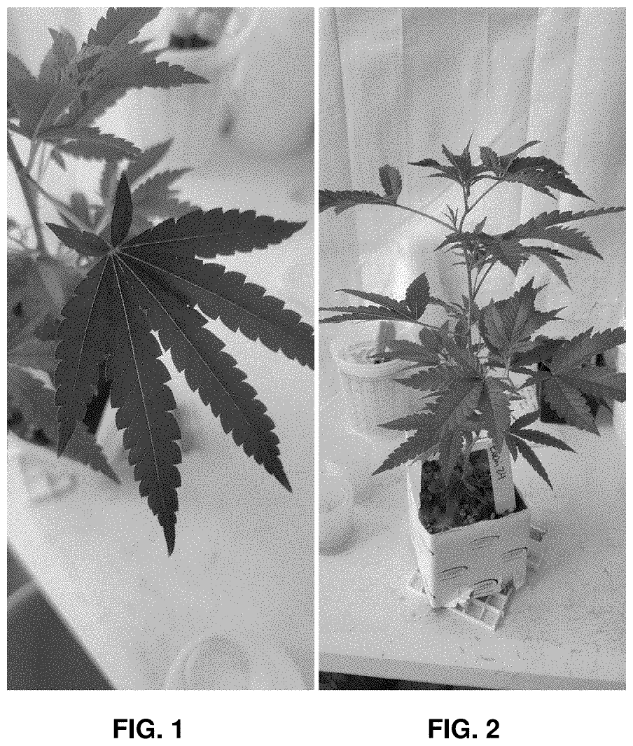

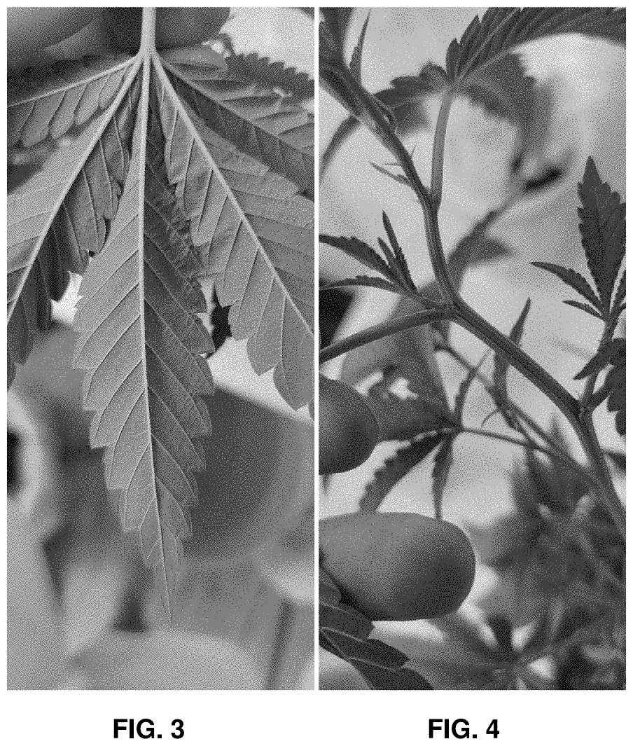

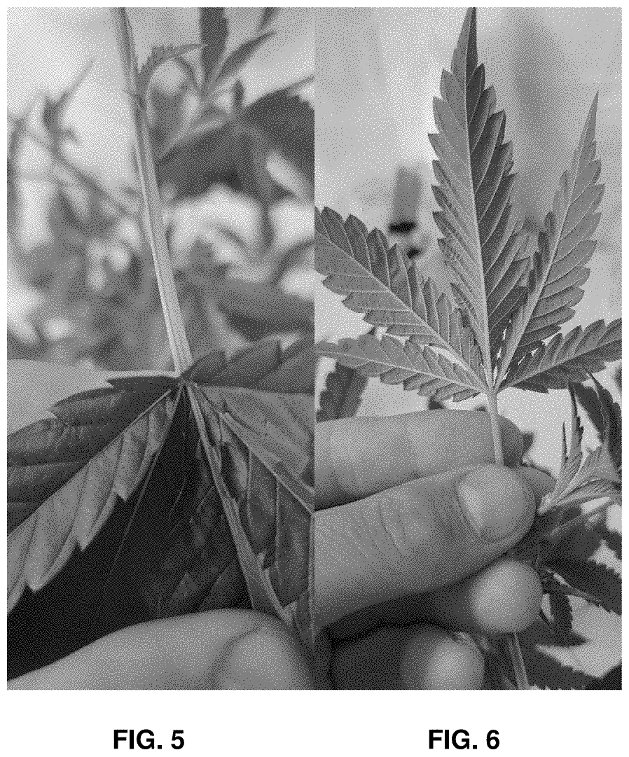

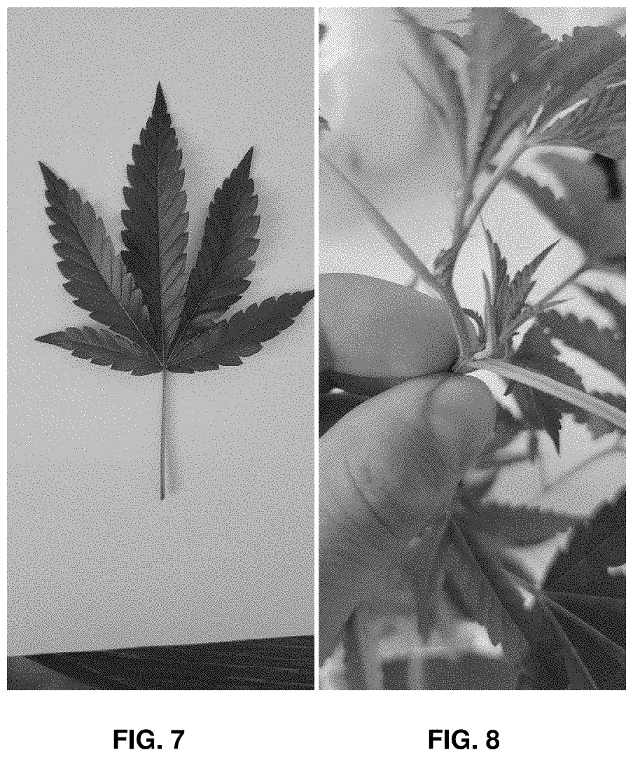

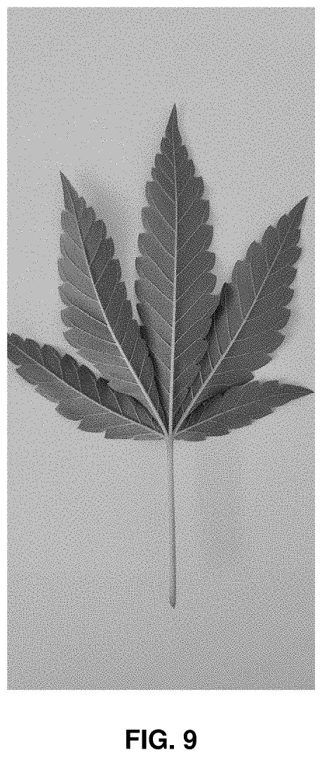

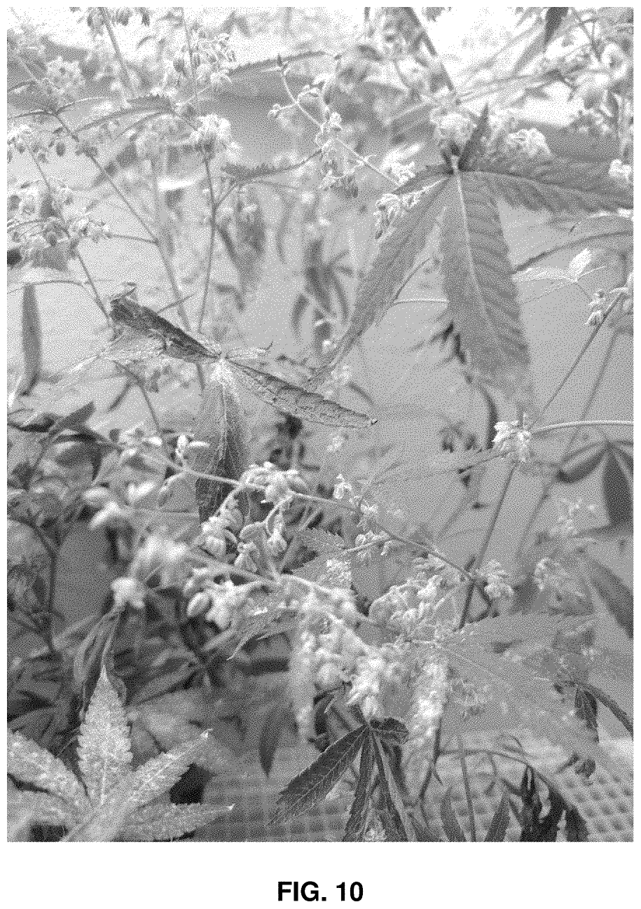

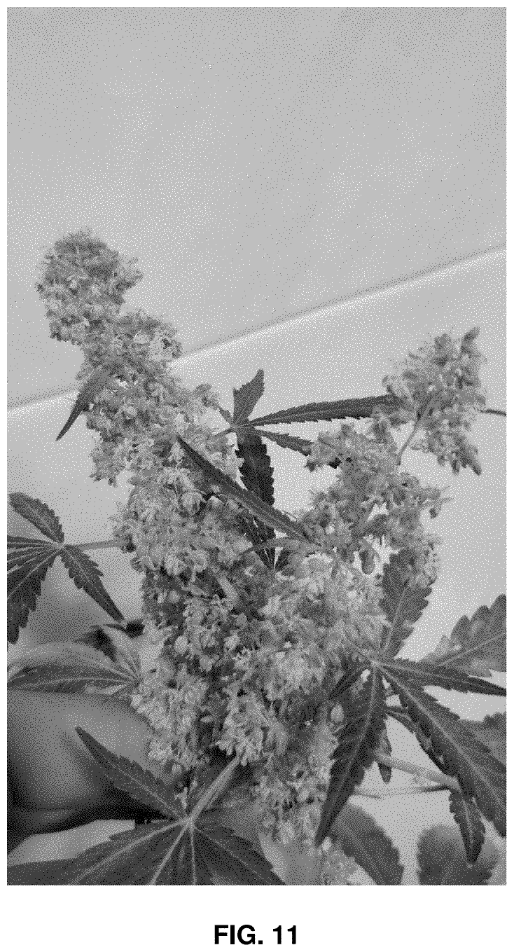

### DETAILED BOTANICAL DESCRIPTION

‘ZUCHI’ has not been observed under all possible environmental conditions, and the phenotype may vary significantly with variations in environment. The following observations, measurements, and comparisons describe this plant as grown at Mentone, Calif., when grown in the greenhouse, nursery or field, unless otherwise noted.

The color chart referenced is standard hexadecimal Web Pantone Color Chart well known to those of ordinary skill in Internet web site design.
The plant
   Type (life form and habit).—Herbaceous tap-rooted annual.
   Classification.—Cultivars of Cannabis sativa, possessing traits of the Subspecies C. sativa ssp. indica (Lam.). This cultivated line possesses intoxicating properties, and so the Subspecies sativa and its varieties (var. sativa and spontanea) are eliminated from consideration. Within the next couplet distinguishing within the Subspecies indica, fruits are required to separate between the varieties (var. indica and var. kafiris tanica). No fruits were found on any of the individuals observed, and so discrimination between the varieties is impossible with this key. Nevertheless, cross-section of the stem revealed that the stem can be hollow depending on the supplemental level of monosilicic acid, a characteristic known to occur with the fiber-producing strains of C. sativa ssp. sativa, and thought to be absent from the intoxicating taxa in the genus. As such, these plants appear to be hybrids of the two subspecies of Cannabis sativa, e.g., C. sativa sativa and C. sativa indica.
Origin, form, and growth characteristics:
   Origin.—Whole Plant Natural Mutation of an F1 cultivar.
   Propagation.—The strain is perpetuated solely by cuttings.
   Mature habit.—Tap-rooted annual, with an extensive fibrous root system, upright and lateral branched aerial portion of the plant. The growth form of all cloned individuals seen (n=10) was highly manipulated by the systematic removal of terminal buds, inducing a greater branching habit Overall size in this form varies in the population from 1-2 m tall and 0.5-1 m across at their widest point. Many petiole and scars on stems from systematic removal of large shade leaves. In this habit, these are very vigorous annual herbs.
   Growth.—Very vigorous annuals herbs.
   Foliage.—Leaves.
   Arrangement.—Alternate, with a zig zag structure.
   Form.—Palmately compound, (3) 5-7 (9) linear-lan ceolate leaflets with glandular hairs.
   Size.—Remaining (those still present when plants were observed) shade leaves, whole.
   Leaf (with petiole).—8-10″ long; middle (largest) leaflet 5-6 inches long, 1.25-2″ wide.
   Margins.—Coarsely serrate.
   Leaf color.—Top — dark green, HEX #234718.
   Leaf color.—Bottom — light green, HEX #377025.
   Veins, bottom.—Pronounced midrib, with straight axial branches at about 45° angle, toward distal end of leaflet.
   Color of bottom veins.—Light green, HEX #ddedde.
   Petiole.—Length: 2-2.5″ at maturity. Color. Light green, HEX #d9f5c1.
   Stipules.— 3/16″ length #7dc449.
   Aroma.—Strongly piquant, with hints of limonene (a cyclic terpene also found in Pinus ponderosa).
   Stem.—Non-hollow, large, rugose, ribbed, with ribs running parallel to stem, 0.5-2″ diameter at base when mature. When stressed red and purple steaking can occur.
   Color.—#dd377c.
   Bottom of stem color.—Light green, Pantone 377 C Color |#7a9a01. A few eglandular branched hairs.
   Height.—1.5-1.9 m at anthesis following heavy pruning regime in cultivation.
Inflorescence:
   Blooming habit.—Elongated thryse, forming large clusters from 0.2-0.5 m in length, densely packed with individual small male calyx subtended by small leaves, these with some observable glandular trichomes.
   Flowers.—Corolla: petals and calyx unified and collectively appressed to and surrounding the ovary.
   Color.—Light green and yellow, HEX Pantone 372.#e4ec8e.
   Diameter.—Individual flowers 1-3 mm, cyme 5-7 cm diameter.
   Shape.—Urceolate (urn-shaped).
   Calyces.—Clusters of male calyx flowers can be observed on all stems and branches during flowering Color: Green, Pantone PMS 364.
   Filaments.—N/A.
   Stigma.—No stigma observed.
   Fruit.—An achene in this genus; however, no fruits were seen.
   Pollen.—Pollen drops from male calyx flowers during the 3rd to 4th week of flowering. Color: Pollen is yellow in color HEX #e9f0a4.
   Petalage.—The plant is essentially without petals (apetalous); these fused and appressed to the base of the ovary with the calyx as the perianth.
   Flowers.—Pedicel. Male calyx are connected to the pedicel that is in turn connected to the peduncle that is in turn connected to the main stem.
   Color.—Pantone 377 C Color |#7a9a01.
General characteristics and culture:
   Blooming period.—Cuttings after rooting will bloom in 3-6 weeks when <12 hrs light applied to induce flowering.
   Hardiness.—Hardiness in nature unknown as this plant has only been cultivated in controlled conditions.
   Breaking action.—Stems are fibrous, strong, and flexible; highly resistant to breakage.
   Rooting.—>95% success rate with cuttings using Dip n Grow rooting solution.
   Growth regulator.—Only naturally occurring cytokines and auxins are used in cultivation. These are derived from kelp, seaweed, and alfalfa extracts. 1-Triacontanol is also used as a biostimulant and is naturally derived from beeswax.
   Shipping tolerance.—Not applicable. This plant has never been shipped and is not intended for live shipment.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A new and distinct cultivar of “Cannabis” plant, as shown and described.
